(19) United States
(12) Patent Application Publication
(10) Pub. No.: US 2025/0017832 A1
(43) Pub. Date: Jan. 16, 2025

(54) EDIBLE CHEW AND METHODS OF USING THE SAME

(71) Applicant: Ark Naturals Company, Tampa, FL (US)

(72) Inventors: Kerri DaSilva, Tampa, FL (US); Michael J. Stoeckle, Tampa, FL (US)

(21) Appl. No.: 18/714,606

(22) PCT Filed: Dec. 1, 2022
(86) PCT No.: PCT/US2022/080715

§ 371 Date: May 30, 2024

Related U.S. Application Data
(60) Provisional application No. 63/284,818, filed on Dec. 1, 2021.

(51) Int. Cl. A61K 8/35; A23K 10/20; A23K 10/30; A23K 20/105; A23K 20/147; A23K 20/158; A23K 20/163; A23K 20/174; A23K 20/189; A23K 20/22; A23K 20/24; A23K 20/26; A23K 40/25; A23K 40/30; A23K 50/40; A61K 8/02; A61K 8/19; A61K 8/24; A61K 8/66; A61K 8/67; A61K 8/92; A61Q 11/00

(52) U.S. Cl. CPC A61K 8/35; A23K 10/20; A23K 10/30; A23K 20/105; A23K 20/147; A23K 20/158; A23K 20/163; A23K 20/174; A23K 20/189; A23K 20/22; A23K 20/24; A23K 20/26; A23K 40/25; A23K 40/30; A23K 50/40; A61K 8/0233; A61K 8/19; A61K 8/24; A61K 8/66; A61K 8/676; A61K 8/922; A61Q 11/00; A61K 2800/28; A61K 2800/92

(57) ABSTRACT

The present invention provides an edible chew for preventing or inhibiting dental plaque accumulation and dental calculus formation in a non-human animal. The edible chew includes a core that includes one or more of a polishing agent, a flavor additive, a vegetable oil, a humectant, a dental additive, a food additive, and water. The edible chew also includes an outer layer disposed on the core. The outer layer includes astaxanthin. The present invention also provides a process for preparing the edible chew. The present invention further provides a method of preventing or inhibiting dental plaque accumulation and/or dental calculus formation in a subject in need thereof.

### CROSS REFERENCE TO RELATED APPLICATION

The present application claims priority to and the benefit of U.S. Provisional Application No. 63/284,818, filed Dec. 1, 2021, the contents of which are herein incorporated by reference in their entirety.

### FIELD OF THE INVENTION

The present invention relates to an edible chew and methods for preventing or inhibiting dental plaque accumulation and/or dental calculus formation in a subject in need thereof.

### BACKGROUND

Non-human animals, such as canines and felines, are susceptible to dental plaque accumulation and/or dental calculus (i.e., tartar) formation. Dental plaque is a soft deposit which forms on the surfaces of teeth. Dental plaque is generally believed to be formed as a byproduct of bacterial growth and comprises a dense microbial layer consisting of a mass of microorganisms embedded in a polysaccharide matrix. Dental plaque adheres to the surfaces of teeth, especially along irregular and rough surfaces, and is typically found at the gingival margin, in cracks in tooth enamel, and on the surface of built-up dental calculus. Dental plaque can give rise to tooth decay and periodontal problems, such as gingivitis and periodontitis.

Dental plaque formed along tooth surfaces provides a locus for dental calculus formation. Dental calculus is a hardened (i.e., mineralized) form of dental plaque resulting from precipitation of minerals from saliva and gingival crevicular fluid (GCF) in dental plaque on teeth. Continued accumulation of both dental plaque and dental calculus can result in gum recession and, ultimately, destruction of bone and periodontal ligament.

Conventional edible chews for non-human animals rely on mechanical removal (e.g., abrasion) to reduce levels of dental plaque and/or dental calculus. In addition, dental plaque and dental calculus may be removed via brushing. While effective at removing dental plaque and/or dental calculus that has already formed and accumulated on teeth of non-human animals, neither conventional chews nor brushing effectively prevent or inhibit dental plaque accumulation and/or dental calculus formation.

As such, there remains a need to provide improved edible chews and methods for preventing or inhibiting dental plaque accumulation and/or dental calculus formation in non-human animals.

### SUMMARY OF THE INVENTION

The present invention provides an edible chew for preventing or inhibiting dental plaque accumulation and dental calculus formation in a non-human animal. The edible chew includes a core that includes one or more of a polishing agent, a flavor additive, a vegetable oil, a humectant, a dental additive, a food additive, and water. The edible chew also includes an outer layer disposed on the core. The outer layer includes astaxanthin.

In some embodiments, the outer layer includes from about 0.05 mg to about 10.0 mg of astaxanthin. In some embodiments, the outer layer includes from about 0.2 mg to about 0.4 mg of astaxanthin. In some embodiments, the outer layer includes from about 0.1 mg to about 0.2 mg of astaxanthin. In some embodiments, the outer layer includes from about 1.5 mg to about 2.0 mg of astaxanthin. In some embodiments, the outer layer includes from about 0.7 mg to about 1.1 mg of astaxanthin. In some embodiments, the outer layer includes from about 2.0 mg to about 3.0 mg of astaxanthin. In some embodiments, the outer layer includes from about 1.0 mg to about 1.5 mg of astaxanthin. In some embodiments, the outer layer includes from about 3.5 mg to about 4.5 mg of astaxanthin. And, in some embodiments, the outer layer includes from about 1.5 mg to about 2.5 mg of astaxanthin.

In some embodiments, the core includes a polishing agent selected from sodium bicarbonate, calcium carbonate, dicalcium phosphate, calcium sulfate, silica, iron oxide, hydroxyapatite, aluminum oxide, perlite, plastic particles, or any combination thereof. In other embodiments, the core includes a polishing agent selected from calcium carbonate, dicalcium phosphate, sodium bicarbonate, or any combination thereof.

In some embodiments, the core includes a flavor additive selected from spearmint, peppermint, wintergreen, sassafras, clove, sage, eucalyptus, marjoram, cinnamon, lemon, lime, grapefruit, coconut, orange, vanilla, rosemary, salt, chicken, honey, or any combination thereof. In other embodiments, the core includes a flavor additive selected from clove, cinnamon, vanilla, rosemary, salt, chicken, honey, or any combination thereof.

In some embodiments, the core includes a vegetable oil selected from palm oil, soybean oil, canola oil, sunflower seed oil, flaxseed oil, peanut oil, cottonseed oil, palm kernel oil, coconut oil, olive oil, or any combination thereof. In other embodiments, the core includes a vegetable oil selected from canola oil, flaxseed oil, or any combination thereof.

In some embodiments, the core includes a humectant selected from glycerin, sorbitol, or any combination thereof. In other embodiments, the humectant is glycerin.

In some embodiments, the core includes a dental additive selected from glucose oxidase, L-ascorbic acid phosphate, lactoperooxidase, tetrasodium pyrophosphate, or any combination thereof. In other embodiments, the core includes a dental additive selected from glucose oxidase, L-ascorbic acid phosphate, tetrasodium pyrophosphate, or any combination thereof.

In some embodiments, the core includes a food additive selected from cultured dextrose, gelatin, lecithin, rice flour, citric acid, or any combination thereof. In some embodiments, the core includes water.

In some embodiments, the core includes one or more binders and/or fillers. In other embodiments, the outer layer includes one or more binders and/or fillers. And, in some embodiments, each of the core and the outer layer independently include one or more binders and/or fillers.

In some embodiments, the outer layer includes an exterior surface facing away from the core that is substantially free of any protrusions configured to remove dental plaque and/or dental calculus from teeth of a subject.

Another aspect of the present invention provides a method of preventing or inhibiting dental plaque accumulation and/or dental calculus formation in a subject in need thereof. The method includes administering to the subject an effective amount of astaxanthin. The subject is a non-human animal.

In some implementations, the astaxanthin is administered at a dose of from about 0.05 mg to about 10 mg at least once per day. In some implementations, the non-human animal has a weight of up to about 8.0 lbs, and the astaxanthin is administered at a dose of from about 0.1 mg to about 0.5 mg at least once per day. In some implementations, the non-human animal has a weight of up to about 8.0 lbs, and the astaxanthin is administered at a dose of from about 0.2 mg to about 0.4 mg once per day. In some implementations, the non-human animal has a weight of up to about 8.0 lbs, and the astaxanthin is administered at a dose of from about 0.1 mg to about 0.2 mg twice per day. In some implementations, the non-human animal has a weight of from about 8.0 lbs to about 20.0 lbs, and the astaxanthin is administered at a dose of from about 0.5 mg to about 2.5 mg at least once per day. In some implementations, the non-human animal has a weight of from about 8.0 lbs to about 20.0 lbs, and the astaxanthin is administered at a dose of from about 1.5 mg to about 2.0 mg once per day. In some implementations, the non-human animal has a weight of from about 8.0 lbs to about 20.0 lbs, and the astaxanthin is administered at a dose of from about 0.7 mg to about 1.1 mg twice per day. In some implementations, the non-human animal has a weight of from about 20.0 lbs to about 40.0 lbs, and the astaxanthin is administered at a dose of from about 1.0 mg to about 3.5 mg at least once per day. In some implementations, the non-human animal has a weight of from about 20.0 lbs to about 40.0 lbs, and the astaxanthin is administered at a dose of from about 2.0 mg to about 3.0 mg once per day. In some implementations, the non-human animal has a weight of from about 20.0 lbs to about 40.0 lbs, and the astaxanthin is administered at a dose of from about 1.0 mg to about 1.5 mg twice per day. In some implementations, the non-human animal has a weight of greater than about 40.0 lbs, and the astaxanthin is administered at a dose of from about 1.5 mg to about 5.0 mg at least once per day. In some implementations, the non-human animal has a weight of greater than about 40.0 lbs, and the astaxanthin is administered at a dose of about 3.5 mg to about 4.5 mg once per day. And, in some implementations, the non-human animal has a weight of greater than about 40.0 lbs, and the astaxanthin is administered at a dose of about 1.5 mg to about 2.5 mg twice per day.

In some implementations, the astaxanthin is administered as an edible chew. In some implementations, the edible chew includes a core and an outer layer disposed on the core. The core includes one or more of a polishing agent, a flavor additive, a vegetable oil, a humectant, a dental additive, a food additive, and water. The outer layer includes the effective amount of astaxanthin.

In some implementations, the non-human animal is at least 12 weeks old. In some implementations, the non-human animal is a canine or a feline. In some implementations, teeth of the non-human animal are cleaned, for example by a veterinarian, prior to the administration of astaxanthin. In some implementations, teeth of the non-human animal are substantially free of dental plaque prior to the administration of astaxanthin. In some implementations, teeth of the non-human animal are substantially free of dental calculus prior to the administration of astaxanthin. In some implementations, the subject experiences an increase in salivary immunoglobulin A (sIgA) concentration following onset of administration.

Another aspect of the present invention provides a method of preventing or inhibiting dental plaque accumulation in a subject in need thereof. The method includes administering to the subject an effective amount of astaxanthin. The subject is a non-human animal.

Another aspect of the present invention provides a method of preventing or inhibiting calculus formation in a subject in need thereof. The method includes administering to the subject an effective amount of astaxanthin. The subject is a non-human animal.

Another aspect of the present invention provides a method of preventing or inhibiting dental plaque accumulation and/or dental calculus formation in a subject in need thereof. The method includes administering to the subject an effective amount of astaxanthin. The astaxanthin is administered as an edible chew. The edible chew includes a core and an outer layer disposed on the core. The core includes one or more of a polishing agent, a flavor additive, a vegetable oil, a humectant, a dental additive, a food additive, and water. The outer layer includes the effective amount of astaxanthin. The subject is a non-human animal. And, the subject experiences an increase in sIgA concentration following onset of administration.

Another aspect of the present invention provides a process for preparing an edible chew for preventing or inhibiting dental plaque accumulation and/or dental calculus formation in a non-human animal. The edible chew includes a core and an outer layer disposed on the core. The core includes one or more of a polishing agent, a flavor additive, a vegetable oil, a humectant, a dental additive, a food additive, and water. The outer layer includes astaxanthin. The process includes extruding the core. The process also includes extruding the outer layer on the core to form the edible chew.

In some implementations, the step of extruding the core and the step of extruding the outer layer are performed simultaneously. In other implementations, the step of extruding the outer layer is performed after the step of extruding the core.

### BRIEF DESCRIPTION OF THE DRAWINGS

The figures below are provided by way of example and are not intended to limit the scope of the claimed invention.

FIG. 1 is a perspective view of an edible chew for preventing or inhibiting dental plaque accumulation and/or dental calculus formation.

FIG. 2 is a transverse cross-sectional view of the edible chew of FIG. 1.

### DETAILED DESCRIPTION

### I. Definitions

As used herein, the term “preventing or inhibiting” with respect to dental plaque accumulation and/or dental calculus formation refers to eliminating dental plaque accumulation and/or dental calculus formation in a subject or reducing dental plaque formation and/or dental calculus formation in a subject. It will be appreciated that dental plaque accumulation and/or dental calculus formation may be inhibited by about 10%, 15%, 20%, 25%, 30%, 35%, 40%, 45%, 50%, 55%, 60%, 65%, 70%, 75%, 80%, 85%, 90%, 95%, 96%, 97%, 97.5%, 98%, 98.5%, 99%, 99.5%, 99.9%, or 99.99% as compared to an untreated subject.

As used herein, the term “core” refers to an edible material that is at least partially surrounded by another layer. In some embodiments, the core may be entirely surrounded by another layer. In some embodiments, the core is chewable.

As used herein, the term “outer layer” refers to an edible material that is disposed on the core and at least partially surrounds the core. In some embodiments, the outer layer entirely surrounds the core. In some embodiments, the outer layer is chewable. In some embodiments, one or more intermediate layers are disposed between the core and the outer layer.

As used herein, the term “about”, when referring to a numerical value or range of values, allows for a degree of variability in the value or range or values, for example, within 10%, or within 5% of a stated value or of a stated limit of a range.

As used herein, the term “subject” to which administration is contemplated includes, but is not limited to, a human and/or a non-human animal, e.g., a mammal such as primates, cattle, pigs, horses, sheep, goats, rodents, cats, and/or dogs. Preferably, the subject is a non-human animal. More preferably, the subject is a companion animal or pet. Even more preferably, the subject is a canine or a feline (e.g., a dog or a cat).

In general, the “effective amount” of a compound (e.g., astaxanthin) refers to an amount sufficient to elicit the desired biological response, e.g., to prevent or inhibit dental plaque accumulation and/or dental calculus formation. As will be appreciated by those of ordinary skill in the art, the effective amount of a compound may vary depending on such factors as the desired biological endpoint, the pharmacokinetics of the compound, the mode of administration, and the age, weight, health, and condition of the subject.

An effective amount can be administered in one or more administrations, applications or dosages. An effective amount can be administered from one or more times per day to one or more times per week; including once every other day. In some embodiments, the effective amount is administered at least once per day. In some embodiments, the effective amount is administered daily before or after a meal, e.g., immediately before or after a meal or 15-60 minutes before or after a meal. In some embodiments, the compositions are administered orally.

In some embodiments, the present invention contemplates administration of an effective amount of the compound (e.g., astaxanthin) as a prophylactic before a subject begins to suffer from dental plaque accumulation or dental calculus formation. As used herein, and unless otherwise specified, a “prophylactically effective amount” of a compound is an amount sufficient to prevent or inhibit dental plaque accumulation and/or dental calculus formation. A prophylactically effective amount of a compound means an amount of a therapeutic agent, alone or in combination with other agents, which provides a prophylactic benefit in the prevention or inhibition of dental plaque accumulation and/or dental calculus formation. The term “prophylactically effective amount” can encompass an amount that improves overall prophylaxis or enhances the prophylactic efficacy of another prophylactic agent.

As used herein, the term “onset of administration” refers to the day on which administration of astaxanthin begins.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

### II. Edible Chew

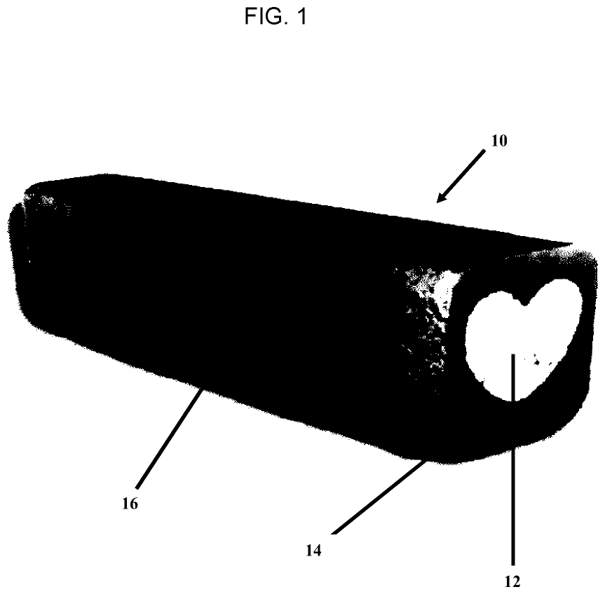

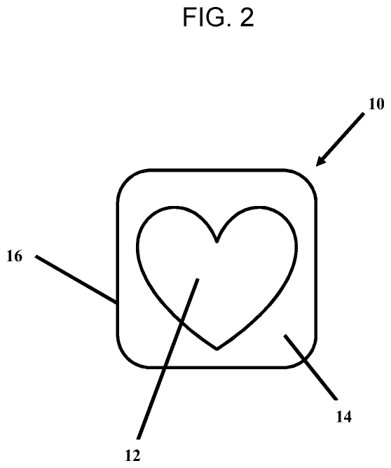

With reference to FIGS. 1 and 2, the present inventions provides an edible chew 10 for preventing or inhibiting dental plaque accumulation and dental calculus formation in a non-human animal. The edible chew includes a core 12 and an outer layer 14 disposed on the core. The core 12 includes one or more of a polishing agent, a flavor additive, a vegetable oil, a humectant, a dental additive, a food additive, and water. The outer layer 14 includes astaxanthin.

### A. Core

With continued reference to FIGS. 1 and 2, the core 12 is an edible material. In some embodiments, the core 12 is chewable. The geometrical configuration of a cross-sectional shape defined by the core 12 is not particularly limited, and may be any cross-sectional shape suitable for consumption by a non-human animal. By way of non-limiting example, the cross-sectional shape defined by the core 12 may be a triangle, square, rectangle, oval, circle, square, diamond, heart, star, bone, or any other geometric shape. As shown in FIG. 2, in some embodiments, the cross-sectional shape defined by the core 12 is a heart.

In some embodiments, the core 12 includes the polishing agent. When present, the polishing agent mechanically removes (i.e., via a polishing action) dental plaque and/or dental calculus from teeth of the non-human animal. The polishing agent is not particular limited, and may be any polishing agent suitable for consumption by a non-human animal that mechanically removes dental plaque and/or dental calculus from teeth. In some embodiments, the core 12 includes a polishing agent selected from sodium bicarbonate, calcium carbonate, dicalcium phosphate, calcium sulfate, silica, iron oxide, hydroxyapatite, aluminum oxide, perlite, plastic particles, or any combination thereof. In some embodiments, the core 12 includes a polishing agent selected from calcium carbonate, dicalcium phosphate, sodium bicarbonate, or any combination thereof. In some embodiments, the polishing agent includes calcium carbonate. In some embodiments, the polishing agent includes dicalcium phosphate. In some embodiments, the polishing agent includes sodium bicarbonate. In some embodiments, the polishing agent includes calcium carbonate, dicalcium phosphate, and sodium bicarbonate. And, in some embodiments, the polishing agent is a combination of calcium carbonate, dicalcium phosphate, and sodium bicarbonate.

In some embodiments, the core 12 includes the flavor additive. When present, the flavor additive improves the palatability of the edible chew 10 and/or freshens breath of the non-human animal upon consumption of the edible chew 10. The flavor additive is not particular limited, and may be any flavor additive suitable for consumption by a non-human animal that is palatable to and/or freshens breath of the non-human animal. In some embodiments, the core 12 includes a flavor additive selected from spearmint, peppermint, wintergreen, sassafras, clove, sage, eucalyptus, marjoram, cinnamon, lemon, lime, grapefruit, coconut, orange, vanilla, rosemary, salt, chicken, honey, or any combination thereof. In some embodiments, the core 12 includes a flavor additive selected from clove, cinnamon, vanilla, rosemary, salt, chicken, honey, or any combination thereof. In some embodiments, the flavor additive includes clove. In some embodiments, the flavor additive includes cinnamon. In some embodiments, the flavor additive includes vanilla. In some embodiments, the flavor additive includes rosemary. In some embodiments, the flavor additive includes salt. In some embodiments, the flavor additive includes chicken. In some embodiments, the flavor additive honey. In some embodiments, the flavor additive includes clove, cinnamon, vanilla, rosemary, salt, chicken, and honey. And, in some embodiments, the flavor additive is a combination of clove, cinnamon, vanilla, rosemary, salt, chicken, and honey.

In some embodiments, the core 12 includes the vegetable oil. The vegetable oil is not particular limited, and may by any vegetable oil suitable for consumption by a non-human animal. In some embodiments, the core 12 includes a vegetable oil selected from palm oil, soybean oil, canola oil, sunflower seed oil, flaxseed oil, peanut oil, cottonseed oil, palm kernel oil, coconut oil, olive oil, or any combination thereof. In some embodiments, the core 12 includes a vegetable oil selected from selected from canola oil, flaxseed oil, or any combination thereof. In some embodiments, the vegetable includes canola oil. In some embodiments, the vegetable oil includes flaxseed oil. In some embodiments, the vegetable oil includes canola oil and flaxseed oil. And, in some embodiments, the vegetable oil is a combination of canola oil and flaxseed oil.

In some embodiments, the core 12 includes the humectant. When present, the humectant retains moisture in the edible chew 10 and may allow for greater control over shape, viscosity, texture, and/or shelf-life of the edible chew 10. The humectant is not particular limited, and may by any humectant suitable for consumption by a non-human animal that retains moisture in the edible chew 10. In some embodiments, the core 12 includes a humectant selected from sucrose, glycerin, glycerol, sorbitol, xylitol, butylene glycol, propylene glycol, or any combination thereof. In some embodiments, the core 12 includes a humectant selected from glycerin, sorbitol, or any combination thereof. In some embodiments, the humectant includes glycerin. In some embodiments, the humectant includes sorbitol. In some embodiments, the humectant includes glycerin and sorbitol. And, in some embodiments, the humectant is glycerin.

In some embodiments, the core 12 includes the dental additive. When present, the dental additive contributes to oral health of teeth of the non-human animal (e.g., anti-dental plaque agents, anti-dental calculus agents, etc.) and/or visual appearance (e.g., whitening) of teeth of the non-human animal. The dental additive is not particularly limited, and may by any dental additive suitable for consumption by a non-human animal that contributes to oral health and/or visual appearance of teeth of the non-human animal. By way of non-limiting example, the dental additive may be one or more of an anti-dental plaque agent, an anti-dental calculus agent, and a whitening agent. In some embodiments, the core 12 includes a dental additive selected from glucose oxidase, L-ascorbic acid phosphate, lactoperooxidase, tetrasodium pyrophosphate, or any combination thereof. In some embodiments, the dental additive includes glucose oxidase. In some embodiments, the dental additive includes L-ascorbic acid phosphate. In some embodiments, the dental additive includes lactoperooxidase. In some embodiments, the dental additive includes tetrasodium pyrophosphate. In some embodiments, the dental additive includes glucose oxidase, L-ascorbic acid phosphate, and tetrasodium pyrophosphate. And, in some embodiments, the dental additive is a combination of glucose oxidase, L-ascorbic acid phosphate, and tetrasodium pyrophosphate.

In some embodiments, the core 12 includes the food additive. When present, the food additive preserves flavor and/or enhances taste, appearance, and/or texture of the edible chew 10. The food additive is not particular limited, and may be any food additive suitable for consumption by a non-human animal that preserves flavor and/or enhances taste, appearance, and/or texture of the edible chew 10. By way of non-limiting example, the food additive may be one or more of an acidulant, an acidity regulator, an anticaking agent, an antifoaming agent, a foaming agent, a bulking agent, an antioxidant, a food coloring, a fortifying agent, a color retention agent, an emulsifier, a flour treatment agent, a glazing agent, a preservative, a stabilizer, and a thickener. In some embodiments, the core 12 includes a food additive selected from cultured dextrose, gelatin, lecithin, rice flour, citric acid, or any combination thereof. In some embodiments, the food additive includes cultured dextrose. In some embodiments, the food additive includes gelatin. In some embodiments, the food additive includes rice flour. In some embodiments, the food additive includes citric acid. In some embodiments, the food additive includes cultured dextrose, gelatin, lecithin, rice flour, and citric acid. And, in some embodiments, the food additive is a combination of cultured dextrose, gelatin, lecithin, rice flour, and citric acid.

In some embodiments, the core 12 includes water. In some embodiments, the core 12 includes the polishing agent, the flavor additive, the vegetable oil, the humectant, the dental additive, the food additive, and water. In some embodiments, the core 12 includes calcium carbonate, canola oil, cinnamon, citric acid, clove, cultured dextrose, dicalcium phosphate, flaxseed oil, gelatin, glucose oxidase, glycerin, honey, L-ascorbic acid phosphate, lecithin, chicken, rice flour, rosemary, salt, sodium bicarbonate, tetrasodium pyrophosphate, vanilla, and water.

In some embodiments, the core 12 includes one or more binders and/or fillers. When present, the binders and/or fillers may be any binders and/or fillers suitable for consumption by a non-human animal. In some embodiments, the core 12 is an extrudate.

In some embodiments, the core 12 includes astaxanthin. In other embodiments, the core 12 is substantially free of astaxanthin.

As will be appreciated by those of ordinary skill in the art, one or more the polishing agents, the flavor additives, the vegetable oils, the humectants, the dental additives, and/or the food additives described herein may also function as another of the polishing agent, the flavor additive, the vegetable oil, the humectant, the dental additive, and/or the food additive. For example, when the flavor additive includes honey, honey may also function as a humectant. As another example, when the humectant includes sorbitol, sorbitol may also function as a flavor additive. As yet another example, when the flavor additive is salt, the salt may serve as a food additive (e.g., a preservative).

### B. Outer Layer

With reference again to FIGS. 1, and 2, the outer layer 14 is an edible material. In some embodiments, the outer layer 14 is chewable. The geometrical configuration of a cross-sectional shape defined by the outer layer 14 is not particularly limited, and may be any cross-sectional shape suitable for consumption by a non-human animal. By way of non-limiting example, the cross-sectional shape defined by the outer layer 14 may be a triangle, square, rectangle, oval, circle, square, diamond, heart, star, bone, or any other shape. As shown in FIG. 2, in some embodiments, the cross-sectional shape defined by the outer layer 14 is a square.

With continued reference to FIGS. 1 and 2, the outer layer 14 includes an exterior surface 16 facing away from the core 12. As shown in FIGS. 1 and 2, the exterior surface 16 may be substantially free of any protrusions configured to remove dental plaque and/or dental calculus from teeth of the non-human animal. In other embodiments, the exterior surface may include protrusions configured to remove dental plaque and/or dental calculus from teeth of the non-human animal. Suitable protrusions for inclusion on the exterior surface 16 of the outer layer 14 are described in U.S. Patent Application Publication No. 2011/0052661, which is hereby incorporated by reference in its entirety.

The outer layer 14 is disposed on the core 12. As shown in FIGS. 1 and 2, in some embodiments, the outer layer 14 surrounds the core 12. In other embodiments, the outer layer 14 partially surrounds the core 12. In other words, there may be portions of the core 12 that the outer layer 14 is not disposed on. In some embodiments, one or more intermediate layers are disposed between the core 12 and the outer layer 14. And, in some embodiments, the outer layer 14 has a red color.

The outer layer 14 includes astaxanthin. In some embodiments, the outer layer 14 includes from about 0.001 mg to about 100.0 mg of astaxanthin. In some embodiments, the outer layer 14 includes from about 0.01 mg about 50.0 mg, about 0.01 mg to about 40.0 mg, about 0.01 mg to about 30.0 mg, about 0.01 mg to about 20.0 mg, or about 0.01 mg to about 10.0 mg of astaxanthin. In some embodiments, the outer layer 14 includes from about 0.05 mg to about 10.0 mg of astaxanthin. In some embodiments, the outer layer 14 includes from about 0.01 mg to about 0.3 mg of astaxanthin. In other embodiments, the outer layer 14 includes from about 0.05 mg to about 0.25 mg of astaxanthin. In some embodiments, the outer layer 14 includes from about 0.1 mg to about 0.2 mg of astaxanthin. In some embodiments, the outer layer 14 includes from about 0.125 mg to about 0.175 mg of astaxanthin. In some embodiments, the outer layer 14 includes from about 0.14 mg to about 0.16 mg of astaxanthin. In some embodiments, the outer layer 14 includes about 0.15 mg of astaxanthin. In some embodiments, the outer layer 14 includes from about 0.1 mg to about 0.5 mg of astaxanthin. In some embodiments, the outer layer 14 includes from about 0.2 mg to about 0.4 mg of astaxanthin. In some embodiments, the outer layer 14 includes from about 0.25 mg to about 0.35 mg of astaxanthin. In some embodiments, the outer layer 14 includes from about 0.275 mg to about 0.325 mg of astaxanthin. In some embodiments, the outer layer 14 includes from about 0.29 mg to about 0.31 mg of astaxanthin. In some embodiments, the outer layer 14 includes about 0.3 mg of astaxanthin. In some embodiments, the outer layer 14 includes from about 0.5 mg to about 1.5 mg of astaxanthin. In some embodiments, the outer layer 14 includes from about 0.75 mg to about 1.25 mg of astaxanthin. In some embodiments, the outer layer 14 includes from about 0.7 mg to about 1.1 mg of astaxanthin. In some embodiments, the outer layer 14 includes from about 0.8 mg to about 1.0 mg of astaxanthin. In some embodiments, the outer layer 14 includes from about 0.85 mg to about 0.95 mg of astaxanthin. In some embodiments, the outer layer 14 includes about 0.9 mg of astaxanthin. In some embodiments, the outer layer 14 includes from about 1.0 mg to about 2.5 mg of astaxanthin. In some embodiments, the outer layer 14 includes from about 1.5 mg to about 2.0 mg of astaxanthin. In some embodiments, the outer layer 14 includes from about 1.6 mg to about 1.95 mg of astaxanthin. In some embodiments, the outer layer 14 includes from about 1.7 mg to about 1.9 mg of astaxanthin. In some embodiments, the outer layer 14 includes from about 1.75 mg to about 1.85 mg of astaxanthin. In some embodiments, the outer layer 14 includes about 1.8 mg of astaxanthin. In some embodiments, the outer layer 14 includes from about 1.5 mg to about 3.5 mg of astaxanthin. In some embodiments, the outer layer 14 includes from about 1.75 mg to about 3.25 mg of astaxanthin. In some embodiments, the outer layer 14 includes from about 2.0 mg to about 3.0 mg of astaxanthin. In some embodiments, the outer layer 14 includes from about 2.2 mg to about 3.0 mg of astaxanthin. In some embodiments, the outer layer 14 includes from about 2.3 mg to about 2.9 mg of astaxanthin. In some embodiments, the outer layer 14 includes from about 2.4 mg to about 2.8 mg of astaxanthin. In some embodiments, the outer layer 14 includes from about 2.5 mg to about 2.7 mg of astaxanthin. In some embodiments, the outer layer 14 includes from about 2.55 mg to about 2.65 mg of astaxanthin. In some embodiments, the outer layer 14 includes about 2.6 mg of astaxanthin. In some embodiments, the outer layer 14 includes from about 0.5 mg to about 2.0 mg of astaxanthin. In some embodiments, the outer layer 14 includes from about 1.0 mg to about 1.5 mg of astaxanthin. In some embodiments, the outer layer 14 includes from about 1.1 mg to about 1.45 mg of astaxanthin. In some embodiments, the outer layer 14 includes from about 1.2 mg to about 1.4 mg of astaxanthin. In some embodiments, the outer layer 14 includes from about 1.25 mg to about 1.35 mg of astaxanthin. In some embodiments, the outer layer 14 includes from about 1.275 mg to about 1.325 mg of astaxanthin. In some embodiments, the outer layer 14 includes about 1.3 mg of astaxanthin. In some embodiments, the outer layer 14 includes from about 3.0 mg to about 5.0 mg of astaxanthin. In some embodiments, the outer layer 14 includes from about 3.5 mg to about 4.5 mg of astaxanthin. In some embodiments, the outer layer 14 includes from about 3.75 mg to about 4.25 mg of astaxanthin. In some embodiments, the outer layer 14 includes from about 3.8 mg to about 4.2 mg of astaxanthin. In some embodiments, the outer layer 14 includes from about 3.9 mg to about 4.1 mg of astaxanthin. In some embodiments, the outer layer 14 includes from about 3.95 mg to about 4.05 mg of astaxanthin. In some embodiments, the outer layer 14 includes about 4.0 mg of astaxanthin. In some embodiments, the outer layer 14 includes from about 1.0 mg to about 3.0 mg of astaxanthin. In some embodiments, the outer layer 14 includes from about 1.25 mg to about 2.75 mg of astaxanthin. In some embodiments, the outer layer 14 includes from about 1.5 mg to about 2.5 mg of astaxanthin. In some embodiments, the outer layer 14 includes from about 1.75 mg to about 2.25 mg of astaxanthin. In some embodiments, the outer layer 14 includes from about 1.8 mg to about 2.2 mg of astaxanthin. In some embodiments, the outer layer 14 includes from about 1.9 mg to about 2.1 mg of astaxanthin. In some embodiments, the outer layer 14 includes from about 1.95 mg to about 2.05 mg of astaxanthin. And, in some embodiments, the outer layer 14 includes about 2.0 mg of astaxanthin.

In some embodiments, the outer layer 14 includes one or more binders and/or fillers. When present, the binders and/or fillers may be any binders and/or fillers suitable for consumption by a non-human animal. In some embodiments, the outer layer 14 is an extrudate. And, in some embodiments, each of the core 12 and the outer layer 14 independently include one or more binders and/or fillers.

In some embodiments, the outer layer 14 includes one or more of the polishing agent, the flavor additive, the vegetable oil, the humectant, the dental additive, the food additive, and water as described herein. In other embodiments, the outer layer 14 is substantially free of any of the polishing agent, the flavor additive, the vegetable oil, the humectant, the dental additive, and the food additive as described herein.

Without wishing to be bound by theory, it is believed that the astaxanthin in the edible chew 10 of the present invention results in an increased concentration of salivary immunoglobulin A (sIgA) in a non-human animal after consumption of the edible chew 10, thereby preventing or inhibiting dental plaque accumulation and/or dental calculus formation. Accordingly, the edible chew 10 is suitable for preventing or inhibiting dental plaque accumulation and/or dental calculus formation in non-human animals. This prevention or inhibition of dental plaque accumulation and/or dental calculus formation is achieved regardless of whether protrusions configured to remove dental plaque and/or dental calculus from teeth of the non-human animal are present on the exterior surface 16 of the outer layer 14. In other words, the edible chew 10 is not reliant on mechanical removal of dental plaque and/or dental calculus in order to prevent or inhibit dental plaque accumulation and/or dental calculus formation in non-human animals.

In some embodiments, the edible chew is free of, or substantially free of, antioxidants other than astaxanthin. In some embodiments, the edible chew is free of, or substantially free of Vitamin E. In some embodiments, the edible chew is free of, or substantially free of Vitamin C. In some embodiments, the ratio of astaxanthin to other antioxidants is 1:1 or greater (e.g., 2:1, 3:1, 5:1, or 10:1). In some embodiments, the ratio of astaxanthin to Vitamin C is 1:1 or greater (e.g., 2:1, 3:1, 5:1, or 10:1). In some embodiments, the ratio of astaxanthin to Vitamin E is 1:1 or greater (e.g., 2:1, 3:1, 5:1, or 10:1).

In some embodiments, the edible chew 10 includes the core 12 and the outer layer 14. The core 12 includes one or more of the polishing agent, the flavor additive, the vegetable oil, the humectant, the dental additive, the food additive, and water. The outer layer 14 includes from about 0.05 to about 10.0 mg of astaxanthin.

In some embodiments, the edible chew 10 includes the core 12 and the outer layer 14. The core 12 includes the polishing agent. The outer layer 14 includes from about 0.05 to about 10.0 mg of astaxanthin. The polishing agent is selected from calcium carbonate, dicalcium phosphate, sodium bicarbonate, or any combination thereof.

In some embodiments, the edible chew 10 includes the core 12 and the outer layer 14. The core 12 includes the dental additive. The outer layer 14 includes from about 0.05 to about 10.0 mg of astaxanthin. The dental additive is selected from glucose oxidase, L-ascorbic acid phosphate, lactoperooxidase, tetrasodium pyrophosphate, or any combination thereof.

In some embodiments, the edible chew 10 includes the core 12 and the outer layer 14. The core 12 includes the polishing agent and the dental additive. The outer layer 14 includes from about 0.05 to about 10.0 mg of astaxanthin. The polishing agent is selected from calcium carbonate, dicalcium phosphate, sodium bicarbonate, or any combination thereof. The dental additive is selected from glucose oxidase, L-ascorbic acid phosphate, lactoperooxidase, tetrasodium pyrophosphate, or any combination thereof.

In some embodiments, the edible chew 10 includes the core 12 and the outer layer 14. The core 12 includes the polishing agent, the flavor additive, the vegetable oil, the humectant, the dental additive, the food additive, and water. The outer layer 14 includes from about 0.05 to about 10.0 mg of astaxanthin.

### 3. Alternative Embodiments

In some embodiments, the edible chew 10 includes one or more intermediate layers disposed between the core 12 and the outer layer 14. When present, the one or more intermediate layers at least partially surround the core 12. In some embodiments, the one or more intermediate layers entirely surround the core 12. In some embodiments, the one or more intermediate layers include one or more of astaxanthin, the polishing agent, the flavor additive, the vegetable oil, the humectant, the dental additive, the food additive, and water as described herein. In other embodiments, the one or more intermediate layers are substantially free of any of astaxanthin, the polishing agent, the flavor additive, the vegetable oil, the humectant, the dental additive, and the food additive as described herein.

Another aspect of the present invention provides an edible chew that includes a core and an outer layer disposed on the core. At least one of the core and the outer layer includes astaxanthin. At least one of the core and the outer layer includes one or more of the polishing agent, the flavor additive, the vegetable oil, the humectant, the dental additive, the food additive, and water as described herein. In some embodiments, the outer layer includes astaxanthin and one or more of the polishing agent, the flavor additive, the vegetable oil, the humectant, the dental additive, the food additive, and water. In some embodiments, the core includes astaxanthin and one or more of the polishing agent, the flavor additive, the vegetable oil, the humectant, the dental additive, the food additive, and water. In other embodiments, the core includes astaxanthin and the outer layer includes one or more of the polishing agent, the flavor additive, the vegetable oil, the humectant, the dental additive, the food additive, and water.

### III. Methods of Preventing or Inhibiting Dental Plaque Accumulation and/or Dental Calculus Formation

Another aspect of the present invention provides a method of preventing or inhibiting dental plaque accumulation and/or dental calculus formation in a subject in need thereof. The method includes administering to the subject an effective amount of astaxanthin. The subject is a non-human animal.

In some implementations, the astaxanthin is administered at a dose of from about 0.001 mg to about 100.0 mg at least once per day. In some implementations, the astaxanthin is administered at a dose of from about 0.01 mg about 50.0 mg, about 0.01 mg to about 40.0 mg, about 0.01 mg to about 30.0 mg, about 0.01 mg to about 20.0 mg, or about 0.01 mg to about 10.0 mg, at least once per day. In some implementations, the astaxanthin is administered at a dose of from about 0.05 mg to about 10.0 mg at least once per day. In some implementations, the astaxanthin is administered at a dose of from about 0.5 mg to about 2.5 mg at least once per day. In some implementations, the astaxanthin is administered at a dose of from about 1.0 mg to about 3.5 mg at least once per day. In some implementations, the astaxanthin is administered at a dose of from about 1.5 mg to about 5.0 mg at least once per day. In some implementations, the astaxanthin is administered at a dose of from about 0.01 mg to about 0.3 mg at least once per day. In some implementations, the astaxanthin is administered at a dose of from about 0.05 mg to about 0.25 mg at least once per day. In some implementations, the astaxanthin is administered at a dose of from about 0.1 mg to about 0.2 mg at least once per day. In some implementations, the astaxanthin is administered at a dose of from about 0.125 mg to about 0.175 mg at least once per day. In some implementations, the astaxanthin is administered at a dose of from about 0.14 mg to about 0.16 mg at least once per day. In some implementations, the astaxanthin is administered at a dose of about 0.15 mg at least once per day. In some implementations, the astaxanthin is administered at a dose of from about 0.1 mg to about 0.5 mg at least once per day. In some implementations, the astaxanthin is administered at a dose of from about 0.2 mg to about 0.4 mg at least once per day. In some implementations, the astaxanthin is administered at a dose of from about 0.25 mg to about 0.35 mg at least once per day. In some implementations, the astaxanthin is administered at a dose of from about 0.275 mg to about 0.325 mg at least once per day. In some implementations, the astaxanthin is administered at a dose of from about 0.275 mg to about 0.325 mg at least once per day. In some implementations, the astaxanthin is administered at a dose of from about 0.29 mg to about 0.31 mg at least once per day. In some implementations, the astaxanthin is administered at a dose of about 0.3 mg at least once per day. In some implementations, the astaxanthin is administered at a dose of from about 0.5 mg to about 1.5 mg at least once per day. In some implementations, the astaxanthin is administered at a dose of from about 0.75 mg to about 1.25 mg at least once per day. In some implementations, the astaxanthin is administered at a dose of from about 0.7 mg to about 1.1 mg at least once per day. In some implementations, the astaxanthin is administered at a dose of from about 0.8 mg to about 1.0 mg at least once per day. In some implementations, the astaxanthin is administered at a dose of from about 0.85 mg to about 0.95 mg at least once per day. In some implementations, the astaxanthin is administered at a dose of about 0.9 mg at least once per day. In some implementations, the astaxanthin is administered at a dose of from about 1.0 mg to about 2.5 mg at least once per day. In some implementations, the astaxanthin is administered at a dose of from about 1.5 mg to about 2.0 mg at least once per day. In some implementations, the astaxanthin is administered at a dose of from about 1.6 mg to about 1.95 mg at least once per day. In some implementations, the astaxanthin is administered at a dose of from about 1.7 mg to about 1.9 mg at least once per day. In some implementations, the astaxanthin is administered at a dose of from about 1.75 mg to about 1.85 mg at least once per day. In some implementations, the astaxanthin is administered at a dose of about 1.8 mg at least once per day. In some implementations, the astaxanthin is administered at a dose of from about 1.5 mg to about 3.5 mg at least once per day. In some implementations, the astaxanthin is administered at a dose of from about 1.75 mg to about 3.25 mg at least once per day. In some implementations, the astaxanthin is administered at a dose of from about 2.0 mg to about 3.0 mg at least once per day. In some implementations, the astaxanthin is administered at a dose of from about 2.2 mg to about 3.0 mg at least once per day. In some implementations, the astaxanthin is administered at a dose of from about 2.3 mg to about 2.9 mg at least once per day. In some implementations, the astaxanthin is administered at a dose of from about 2.4 mg to about 2.8 mg at least once per day. In some implementations, the astaxanthin is administered at a dose of from about 2.5 mg to about 2.7 mg at least once per day. In some implementations, the astaxanthin is administered at a dose of from about 2.55 mg to about 2.65 mg at least once per day. In some implementations, the astaxanthin is administered at a dose of about 2.6 mg at least once per day. In some implementations, the astaxanthin is administered at a dose of from about 0.5 mg to about 2.0 mg at least once per day. In some implementations, the astaxanthin is administered at a dose of from about 1.0 mg to about 1.5 mg at least once per day. In some implementations, the astaxanthin is administered at a dose of from about 1.1 mg to about 1.45 mg at least once per day. In some implementations, the astaxanthin is administered at a dose of from about 1.2 mg to about 1.4 mg at least once per day. In some implementations, the astaxanthin is administered at a dose of from about 1.25 mg to about 1.35 mg at least once per day. In some implementations, the astaxanthin is administered at a dose of from about 1.275 mg to about 1.325 mg at least once per day. In some implementations, the astaxanthin is administered at a dose of about 1.3 mg at least once per day. In some implementations, the astaxanthin is administered at a dose of from about 3.0 mg to about 5.0 mg at least once per day. In some implementations, the astaxanthin is administered at a dose of from about 3.5 mg to about 4.5 mg at least once per day. In some implementations, the astaxanthin is administered at a dose of from about 3.75 mg to about 4.25 mg at least once per day. In some implementations, the astaxanthin is administered at a dose of from about 3.8 mg to about 4.2 mg at least once per day. In some implementations, the astaxanthin is administered at a dose of from about 3.9 mg to about 4.1 mg at least once per day. In some implementations, the astaxanthin is administered at a dose of from about 3.95 mg to about 4.05 mg at least once per day. In some implementations, the astaxanthin is administered at a dose of about 4.0 mg at least once per day. In some implementations, the astaxanthin is administered at a dose of from about 1.0 mg to about 3.0 mg at least once per day. In some implementations, the astaxanthin is administered at a dose of from about 1.25 mg to about 2.75 mg at least once per day. In some implementations, the astaxanthin is administered at a dose of from about 1.5 mg to about 2.5 mg at least once per day. In some implementations, the astaxanthin is administered at a dose of from about 1.75 mg to about 2.25 mg at least once per day. In some implementations, the astaxanthin is administered at a dose of from about 1.8 mg to about 2.2 mg at least once per day. In some implementations, the astaxanthin is administered at a dose of from about 1.9 mg to about 2.1 mg at least once per day. In some implementations, the astaxanthin is administered at a dose of from about 1.95 mg to about 2.05 mg at least once per day. And, in some implementations, the astaxanthin is administered at a dose of about 2.0 mg at least once per day.

In some implementations, the non-human animal has a weight of up to about 8.0 lbs. In some implementations, the non-human animal has a weight of from about 8.0 lbs to about 20.0 lbs. In some implementations, the non-human animal has a weight of from about 20.0 lbs to about 40.0 lbs. In other implementations, the non-human animal has a weight of greater than about 40.0 lbs. For example, the non-human animal may have a weight of from about 40.0 lbs to about 350 lbs.

In some implementations, the astaxanthin is administered at a dose based on the weight of the non-human animal. In some implementations, the non-human animal has a weight of up to about 8.0 lbs, and the astaxanthin is administered at a dose of from about 0.1 mg to about 0.5 mg at least once per day. In some implementations, the non-human animal has a weight of up to about 8.0 lbs, and the astaxanthin is administered at a dose of from about 0.2 mg to about 0.4 mg once per day. In some implementations, the non-human animal has a weight of up to about 8.0 lbs, and the astaxanthin is administered at a dose of from about 0.25 mg to about 0.35 mg once per day. In some implementations, the non-human animal has a weight of up to about 8.0 lbs, and the astaxanthin is administered at a dose of from about 0.275 mg to about 0.325 mg once per day. In some implementations, the non-human animal has a weight of up to about 8.0 lbs, and the astaxanthin is administered at a dose of from about 0.275 mg to about 0.325 mg once per day. In some implementations, the non-human animal has a weight of up to about 8.0 lbs, and the astaxanthin is administered at a dose of from about 0.29 mg to about 0.31 mg once per day. And, in some implementations, the non-human animal has a weight of up to about 8.0 lbs, and the astaxanthin is administered at a dose of about 0.3 mg once per day.

In some implementations, the non-human animal has a weight of up to about 8.0 lbs, and the astaxanthin is administered at a dose of from about 0.01 mg to about 0.3 mg twice per day. In some implementations, the non-human animal has a weight of up to about 8.0 lbs, and the astaxanthin is administered at a dose of from about 0.05 mg to about 0.25 mg twice per day. In some implementations, the non-human animal has a weight of up to about 8.0 lbs, and the astaxanthin is administered at a dose of from about 0.1 mg to about 0.2 mg twice per day. In some implementations, the non-human animal has a weight of up to about 8.0 lbs, and the astaxanthin is administered at a dose of from about 0.125 mg to about 0.175 mg twice per day. In some implementations, the non-human animal has a weight of up to about 8.0 lbs, and the astaxanthin is administered at a dose of from about 0.14 mg to about 0.16 mg twice per day. And, in some implementations, the non-human animal has a weight of up to about 8.0 lbs, and the astaxanthin is administered at a dose of about 0.15 mg twice per day.

In some implementations, the non-human animal has a weight of from about 8.0 lbs to about 20.0 lbs, and the astaxanthin is administered at a dose of from about 0.5 mg to about 2.5 mg at least once per day. In some implementations, the non-human animal has a weight of from about 8.0 lbs to about 20.0 lbs, and the astaxanthin is administered at a dose of from about 1.0 mg to about 2.5 mg once per day. In some implementations, the non-human animal has a weight of from about 8.0 lbs to about 20.0 lbs, and the astaxanthin is administered at a dose of from about 1.5 mg to about 2.0 mg once per day. In some implementations, the non-human animal has a weight of from about 8.0 lbs to about 20.0 lbs, and the astaxanthin is administered at a dose of from about 1.6 mg to about 1.95 mg once per day. In some implementations, the non-human animal has a weight of from about 8.0 lbs to about 20.0 lbs, and the astaxanthin is administered at a dose of from about 1.7 mg to about 1.9 mg once per day. In some implementations, the non-human animal has a weight of from about 8.0 lbs to about 20.0 lbs, and the astaxanthin is administered at a dose of from about 1.75 mg to about 1.85 mg once per day. And, in some implementations, the non-human animal has a weight of from about 8.0 lbs to about 20.0 lbs, and the astaxanthin is administered at a dose of about 1.8 mg once per day.

In some implementations, the non-human animal has a weight of from about 8.0 lbs to about 20.0 lbs, and the astaxanthin is administered at a dose of from about 0.5 mg to about 1.5 mg twice per day. In some implementations, the non-human animal has a weight of from about 8.0 lbs to about 20.0 lbs, and the astaxanthin is administered at a dose of from about 0.75 mg to about 1.25 mg twice per day. In some implementations, the non-human animal has a weight of from about 8.0 lbs to about 20.0 lbs, and the astaxanthin is administered at a dose of from about 0.7 mg to about 1.1 mg twice per day. In some implementations, the non-human animal has a weight of from about 8.0 lbs to about 20.0 lbs, and the astaxanthin is administered at a dose of from about 0.8 mg to about 1.0 mg twice per day. In some implementations, the non-human animal has a weight of from about 8.0 lbs to about 20.0 lbs, and the astaxanthin is administered at a dose of from about 0.85 mg to about 0.95 mg twice per day. And, in some implementations, the non-human animal has a weight of from about 8.0 lbs to about 20.0 lbs, and the astaxanthin is administered at a dose of about 0.9 mg twice per day.

In some implementations, the non-human animal has a weight of from about 20.0 lbs to about 40.0 lbs, and the astaxanthin is administered at a dose of from about 1.0 mg to about 3.5 mg at least once per day. In some implementations, the non-human animal has a weight of from about 20.0 lbs to about 40.0 lbs, and the astaxanthin is administered at a dose of from about 1.5 mg to about 3.5 mg at least once per day. In some implementations, the non-human animal has a weight of from about 20.0 lbs to about 40.0 lbs, and the astaxanthin is administered at a dose of from about 1.75 mg to about 3.25 mg once per day. In some implementations, the non-human animal has a weight of from about 20.0 lbs to about 40.0 lbs, and the astaxanthin is administered at a dose of from about 2.0 mg to about 3.0 mg once per day. In some implementations, the non-human animal has a weight of from about 20.0 lbs to about 40.0 lbs, and the astaxanthin is administered at a dose of from about 2.2 mg to about 3.0 mg once per day. In some implementations, the non-human animal has a weight of from about 20.0 lbs to about 40.0 lbs, and the astaxanthin is administered at a dose of from about 2.3 mg to about 2.9 mg once per day. In some implementations, the non-human animal has a weight of from about 20.0 lbs to about 40.0 lbs, and the astaxanthin is administered at a dose of from about 2.4 mg to about 2.8 mg once per day. In some implementations, the non-human animal has a weight of from about 20.0 lbs to about 40.0 lbs, and the astaxanthin is administered at a dose of from about 2.5 mg to about 2.7 mg once per day. In some implementations, the non-human animal has a weight of from about 20.0 lbs to about 40.0 lbs, and the astaxanthin is administered at a dose of from about 2.55 mg to about 2.65 mg once per day. In some implementations, the non-human animal has a weight of from about 20.0 lbs to about 40.0 lbs, and the astaxanthin is administered at a dose of about 2.6 mg once per day.

In some implementations, the non-human animal has a weight of from about 20.0 lbs to about 40.0 lbs, and the astaxanthin is administered at a dose of from about 0.5 mg to about 2.0 mg twice per day. In some implementations, the non-human animal has a weight of from about 20.0 lbs to about 40.0 lbs, and the astaxanthin is administered at a dose of from about 1.0 mg to about 1.5 mg twice per day. In some implementations, the non-human animal has a weight of from about 20.0 lbs to about 40.0 lbs, and the astaxanthin is administered at a dose of from about 1.1 mg to about 1.45 mg twice per day. In some implementations, the non-human animal has a weight of from about 20.0 lbs to about 40.0 lbs, and the astaxanthin is administered at a dose of from about 1.2 mg to about 1.4 mg twice per day. In some implementations, the non-human animal has a weight of from about 20.0 lbs to about 40.0 lbs, and the astaxanthin is administered at a dose of from about 1.25 mg to about 1.35 mg twice per day. In some implementations, the non-human animal has a weight of from about 20.0 lbs to about 40.0 lbs, and the astaxanthin is administered at a dose of from about 1.275 mg to about 1.325 mg twice per day. And, in some implementations, the non-human animal has a weight of from about 20.0 lbs to about 40.0 lbs, and the astaxanthin is administered at a dose of about 1.3 mg twice per day.

In some implementations, the non-human animal has a weight of greater than about 40.0 lbs, and the astaxanthin is administered at a dose of from about 1.5 mg to about 5.0 mg at least once per day. In some implementations, the non-human animal has a weight of greater than about 40.0 lbs, and the astaxanthin is administered at a dose of from about 3.0 mg to about 5.0 mg once per day. In some implementations, the non-human animal has a weight of greater than about 40.0 lbs, and the astaxanthin is administered at a dose of from about 3.5 mg to about 4.5 mg once per day. In some implementations, the non-human animal has a weight of greater than about 40.0 lbs, and the astaxanthin is administered at a dose of from about 3.75 mg to about 4.25 mg once per day. In some implementations, the non-human animal has a weight of greater than about 40.0 lbs, and the astaxanthin is administered at a dose of from about 3.8 mg to about 4.2 mg once per day. In some implementations, the non-human animal has a weight of greater than about 40.0 lbs, and the astaxanthin is administered at a dose of from about 3.9 mg to about 4.1 mg once per day. In some implementations, the non-human animal has a weight of greater than about 40.0 lbs, and the astaxanthin is administered at a dose of from about 3.95 mg to about 4.05 mg once per day. In some implementations, the non-human animal has a weight of greater than about 40.0 lbs, and the astaxanthin is administered at a dose of about 4.0 mg once per day.

In some implementations, the non-human animal has a weight of greater than about 40.0 lbs, and the astaxanthin is administered at a dose of from about 1.0 mg to about 3.0 mg twice per day. In some implementations, the non-human animal has a weight of greater than about 40.0 lbs, and the astaxanthin is administered at a dose of from about 1.25 mg to about 2.75 mg twice per day. In some implementations, the non-human animal has a weight of greater than about 40.0 lbs, and the astaxanthin is administered at a dose of from about 1.5 mg to about 2.5 mg twice per day. In some implementations, the non-human animal has a weight of greater than about 40.0 lbs, and the astaxanthin is administered at a dose of from about 1.75 mg to about 2.25 mg twice per day. In some implementations, the non-human animal has a weight of greater than about 40.0 lbs, and the astaxanthin is administered at a dose of from about 1.8 mg to about 2.2 mg twice per day. In some implementations, the non-human animal has a weight of greater than about 40.0 lbs, and the astaxanthin is administered at a dose of from about 1.9 mg to about 2.1 mg twice per day. In some implementations, the non-human animal has a weight of greater than about 40.0 lbs, and the astaxanthin is administered at a dose of from about 1.95 mg to about 2.05 mg twice per day. And, in some implementations, the non-human animal has a weight of greater than about 40.0 lbs, and the astaxanthin is administered at a dose of about 2.0 mg twice per day.

In some implementations, the astaxanthin is administered orally, parenterally, by inhalation spray, topically, rectally, nasally, buccally, vaginally or via an implanted reservoir. In other implementations, the astaxanthin is administered orally. In some implementations, the astaxanthin is administered as the edible chew 10 described herein. In some implementations, the astaxanthin is administered as the edible chew 10 described herein at least once per day. And, in some implementations, the astaxanthin is administered as the edible chew 10 described herein twice per day.

In some implementations, the non-human animal is at least 12 weeks old. In other implementations, the non-human animal is from about 0 to about 12 weeks old.

In some implementations, the non-human animal is a canine (e.g., a dog) or a feline (e.g., a cat). In some implementations, the non-human animal is a dog. In some implementations, teeth of the non-human animal are cleaned by a veterinarian prior to the administration of astaxanthin. In some implementations, teeth of the non-human animal are substantially free of dental plaque prior to the administration of astaxanthin. In some implementations, teeth of the non-human animal are substantially free of dental calculus prior to the administration of astaxanthin. In other implementations, teeth of the non-human animal are not cleaned prior to the administration of astaxanthin.

In some implementations, the subject (i.e., the non-human animal) experiences an increase in salivary immunoglobulin A (sIgA) concentration following onset of administration. In some implementations, the subject experiences an increase in sIgA concentration of at least about 1% following onset of administration. In other implementations, the subject experiences an increase in sIgA concentration of at least about 2.5% following onset of administration. In some implementations, the subject experiences an increase in sIgA concentration of at least about 5% following onset of administration. In some implementations, the subject experiences an increase in sIgA concentration of at least about 10% following onset of administration. And, in some implementations, the subject experiences an increase in sIgA concentration of at least about 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 60%, 70%, 80%, 90%, or 99% following onset of administration.

In some implementations, the subject experiences an increase in sIgA concentration about 1 day following onset of administration. In some implementations, the subject experiences an increase in sIgA concentration about 2 days following onset of administration. In some implementations, the subject experiences an increase in sIgA concentration about 5 days following onset of administration. In some implementations, the subject experiences an increase in sIgA concentration about 10 days following onset of administration. In some implementations, the subject experiences an increase in sIgA concentration about 20 days following onset of administration. In some implementations, the subject experiences an increase in sIgA concentration about 30 days following onset of administration. In some implementations, the subject experiences an increase in sIgA concentration about 50 days following onset of administration. In some implementations, the subject experiences an increase in sIgA concentration about 60 days following onset of administration. In some implementations, the subject experiences an increase in sIgA concentration about 90 days following onset of administration.

In some implementations, the subject does not experience a reduction in sIgA concentration when exposed to increased levels of stress (e.g., physical and/or mental stress) following onset of administration. In other implementations, the subject experiences a reduced reduction in sIgA concentration when exposed to increased levels of stress following onset of administration as compared to a subject that is not administered astaxanthin.

In some implementations, the subject experiences reduced dental plaque accumulation as compared to a subject that is not administered astaxanthin. In other implementations, the subject experiences reduced dental calculus formation as compared to a subject that is not administered astaxanthin.

In some implementations, astaxanthin is administered at least once per day for about 2 days. In some implementations, astaxanthin is administered at least once per day for about 5 days. In some implementations, astaxanthin is administered at least once per day for about 10 days. In some implementations, astaxanthin is administered at least once per day for about 20 days. In some implementations, astaxanthin is administered at least once per day for about 30 days. In some implementations, astaxanthin is administered at least once per day for about 50 days. In some implementations, astaxanthin is administered at least once per day for about 60 days. In some implementations, astaxanthin is administered at least once per day for about 90 days. In some implementations, astaxanthin is administered at least once per day for about 1 year. In some implementations, astaxanthin is administered twice per day for about 2 days. In some implementations, astaxanthin is administered twice per day for about 5 days. In some implementations, astaxanthin is administered twice per day for about 10 days. In some implementations, astaxanthin is administered twice per day for about 20 days. In some implementations, astaxanthin is administered twice per day for about 30 days. In some implementations, astaxanthin is administered twice per day for about 50 days. In some implementations, astaxanthin is administered twice per day for about 60 days. In some implementations, astaxanthin is administered twice per day for about 90 days. And, in some implementations, astaxanthin is administered twice per day for about 1 year.

In some implementations, the method of preventing or inhibiting dental plaque accumulation and/or dental calculus formation includes administering to the subject the effective amount of astaxanthin. The subject is a non-human animal. The astaxanthin is administered at a dose of from about 0.05 mg to about 10 mg at least once per day.

In some implementations, the method of preventing or inhibiting dental plaque accumulation and/or dental calculus formation includes administering to the subject the effective amount of astaxanthin. The subject is a non-human animal. The astaxanthin is administered at a dose of from about 0.05 mg to about 10 mg twice per day.

In some implementations, the method of preventing or inhibiting dental plaque accumulation and/or dental calculus formation includes administering to the subject the effective amount of astaxanthin. The subject is a canine or a feline. The astaxanthin is administered at a dose of from about 0.05 mg to about 10 mg at least once per day. The astaxanthin is administered as the edible chew 10. The edible chew 10 includes the core 12 and the outer layer 14. The core 12 includes one or more of the polishing agent, the flavor additive, the vegetable oil, the humectant, the dental additive, the food additive, and water. The outer layer 14 includes the effective amount of astaxanthin. The subject experiences an increase in sIgA concentration following onset of administration.

In some implementations, the method of preventing or inhibiting dental plaque accumulation and/or dental calculus formation includes administering to the subject the effective amount of astaxanthin. The subject is a canine or a feline. The astaxanthin is administered at a dose of from about 0.05 mg to about 10 mg at least once per day. The astaxanthin is administered as the edible chew 10. The edible chew 10 includes the core 12 and the outer layer 14. The core 12 includes the polishing agent. The polishing agent is selected from calcium carbonate, dicalcium phosphate, sodium bicarbonate, or any combination thereof. The outer layer 14 includes the effective amount of astaxanthin. The subject experiences an increase in sIgA concentration following onset of administration.

In some implementations, the method of preventing or inhibiting dental plaque accumulation and/or dental calculus formation includes administering to the subject the effective amount of astaxanthin. The subject is a canine or a feline. The astaxanthin is administered at a dose of from about 0.05 mg to about 10 mg at least once per day. The astaxanthin is administered as the edible chew 10. The edible chew 10 includes the core 12 and the outer layer 14. The core 12 includes the dental additive. The dental additive is selected from glucose oxidase, L-ascorbic acid phosphate, lactoperooxidase, tetrasodium pyrophosphate, or any combination thereof. The outer layer 14 includes the effective amount of astaxanthin. The subject experiences an increase in sIgA concentration following onset of administration.

In some implementations, the method of preventing or inhibiting dental plaque accumulation and/or dental calculus formation includes administering to the subject the effective amount of astaxanthin. The subject is a canine or a feline. The astaxanthin is administered at a dose of from about 0.05 mg to about 10 mg at least once per day. The astaxanthin is administered as the edible chew 10. The edible chew 10 includes the core 12 and the outer layer 14. The core 12 includes the polishing agent and the dental additive. The polishing agent is selected from calcium carbonate, dicalcium phosphate, sodium bicarbonate, or any combination thereof. The dental additive is selected from glucose oxidase, L-ascorbic acid phosphate, lactoperooxidase, tetrasodium pyrophosphate, or any combination thereof. The outer layer 14 includes the effective amount of astaxanthin. The subject experiences an increase in sIgA concentration following onset of administration.

In some implementations, the method of preventing or inhibiting dental plaque accumulation and/or dental calculus formation includes administering to the subject the effective amount of astaxanthin. The subject is a canine or a feline. The astaxanthin is administered at a dose of from about 0.05 mg to about 10 mg at least once per day. The astaxanthin is administered as the edible chew 10. The edible chew 10 includes the core 12 and the outer layer 14. The core 12 includes the polishing agent, the flavor additive, the vegetable oil, the humectant, the dental additive, the food additive, and water. The outer layer 14 includes the effective amount of astaxanthin. The subject experiences an increase in sIgA concentration following onset of administration.

Another aspect of the invention provides a method of preventing or inhibiting dental plaque accumulation in a subject in need thereof. The method includes administering astaxanthin as described herein. The subject is as described herein.

Another aspect of the invention provides a method of preventing or inhibiting dental calculus formation in a subject in need thereof. The method includes administering astaxanthin as described herein. The subject is as described herein.

Another aspect of the invention provides a method of preventing or inhibiting dental plaque accumulation and dental calculus formation in a subject in need thereof. The method includes administering astaxanthin as described herein. The subject is as described herein.

### IV. Process for Preparing an Edible Chew

Another aspect of the present invention provides a process for preparing an edible chew 10 as described herein. The edible chew 10 includes the core 12 and an outer layer 14 disposed on the core 12. The process includes extruding the core 12. The process also includes extruding the outer layer 14 on the core 12 to form the edible chew 10.

In some implementations, the step of extruding the core 12 and the step of extruding the outer layer 14 are performed simultaneously. In other implementations, the step of extruding the outer layer 14 is performed after the step of extruding the core 12.

In some implementations, the step of extruding the core 12 and the step of extruding the outer layer 14 are performed by a single screw extruder. In other implementations, the step of extruding the core 12 and the step of extruding the outer layer 14 are preformed by a twin-screw extruder.

In some implementations, the step of extruding the core 12 further includes cooking a core composition and extruding the core composition to form the core 12. In some implementations, the steps of cooking the core composition and extruding the core composition are performed simultaneously. In other implementations, the step of extruding the core composition is performed after the step of cooking the core composition. In some implementations, the steps of cooking the core composition and extruding the core composition are performed in a single extruder. In other implementations, the step of cooking the core composition is performed in a first extruder and the step of extruding the core composition is performed in a second extruder.

In some implementations, the step of extruding the outer layer 14 further includes cooking an outer layer composition and extruding the outer layer composition to form the outer layer 14. In some implementations, the steps of cooking the outer layer composition and extruding the outer layer composition are performed simultaneously. In other implementations, the step of extruding the outer layer composition is performed after the step of cooking the outer layer composition. In some implementations, the steps of cooking the outer layer composition and extruding the outer layer composition are performed in a single extruder. In other implementations, the step of cooking the outer layer composition is performed in a first extruder and the step of extruding the outer layer composition is performed in a second extruder.

### V. EXAMPLES

In order that the invention described herein may be more fully understood, the following examples are set forth. The examples described in this application are offered to illustrate the edible chews and methods provided herein and are not to be construed in any way as limiting their scope.

### Example 1: Exemplary Edible Chews

Exemplary edible chews are set forth in Table 1 below and were prepared according to processes described herein.

TABLE 1
Exemplary edible chews

| | | Intended non- |
|---|---|---|
| Example | | human animal |
| no. | Components Include | weight |
| 1 | Astaxanthin (0.15 mg), calcium carbonate, canola oil, | Up to 8.0 lbs |
| | cinnamon, citric acid, clove, cultured dextrose, dicalcium | |
| | phosphate, flaxseed oil, gelatin, glucose oxidase, glycerin, | |
| | honey, L-ascorbic acid phosphate, lecithin, chicken, rice | |
| | flour, rosemary, salt, sodium bicarbonate, tetrasodium | |
| | pyrophosphate, vanilla, and water | |
| 2 | Astaxanthin (0.9 mg), calcium carbonate, canola oil, | 8.0 lbs to 20.0 |
| | cinnamon, citric acid, clove, cultured dextrose, dicalcium | lbs |
| | phosphate, flaxseed oil, gelatin, glucose oxidase, glycerin, | |
| | honey, L-ascorbic acid phosphate, lecithin, chicken, rice | |
| | flour, rosemary, salt, sodium bicarbonate, tetrasodium | |
| | pyrophosphate, vanilla, and water | |
| 3 | Astaxanthin (1.3 mg), calcium carbonate, canola oil, | 20.0 lbs to 40.0 |
| | cinnamon, citric acid, clove, cultured dextrose, dicalcium | lbs |
| | phosphate, flaxseed oil, gelatin, glucose oxidase, glycerin, | |
| | honey, L-ascorbic acid phosphate, lecithin, chicken, rice | |
| | flour, rosemary, salt, sodium bicarbonate, tetrasodium | |
| | pyrophosphate, vanilla, and water | |
| 4 | Astaxanthin (2.0 mg), calcium carbonate, canola oil, | Greater than 40.0 |
| | cinnamon, citric acid, clove, cultured dextrose, dicalcium | lbs |
| | phosphate, flaxseed oil, gelatin, glucose oxidase, glycerin, | |
| | honey, L-ascorbic acid phosphate, lecithin, chicken, rice | |
| | flour, rosemary, salt, sodium bicarbonate, tetrasodium | |
| | pyrophosphate, vanilla, and water | |

### Example 2: Effect of Edible Chew on Oral Health in Dogs

The objective of this study is to evaluate the effect of an edible chew according to the present invention (“test treat”) on oral health in dogs. The test treat may optionally be an edible chew as described in Example 1.

The study will be 44 days in length (Preload: Days −14 to 0, Test: 1 to 27). On Days −14 through 27 inclusive, all dogs assigned to Group 1 will be fed control diet, a kibble, fed dry, only. Dogs assigned to Group 2 will be offered the control diet, a kibble, fed dry, and one test treat. A control diet of Purina Dog Chow will be used. On Day 0, each animal will have saliva collected for microbiome analysis, followed by halitosis and gingivitis scoring. After halitosis and gingivitis assessment is complete, plaque will be collected for microbiome analysis, then the teeth will be scaled and polished. After the cleaning procedure, a disclosing agent of 2% Eosin (red dye) will be applied to all test teeth and an oral examination will be performed to ensure there is no remaining plaque or calculus buildup and a clean mouth is used for the start of the test phase. On Day 28, each animal will have saliva collected for microbiome analysis and then undergo halitosis, gingivitis, and plaque evaluation. Plaque will once again be collected for microbiome analysis from the target teeth after disclosure for scoring purposes and prior to being brushed off for calculus evaluation.

Healthy adult beagle dogs will be eligible for the study. Before study initiation, the dogs will undergo an oral cavity examination for normal occlusion and the presence of all test teeth. The initial oral examination will include a visual check for the presence of a significant furcation defect (loss of bone between the roots of multi-rooted teeth). Dogs with any complete furcation defect (i.e., a periodontal probe can be passed from the buccal surface and seen to emerge on the palatal/lingual surface) in any of required test teeth will not be included in this study.

### Preloading Phase (Day −14 to Day 0):

During the pre-test phase of the study (Days −14-0), dogs assigned to Group 1 will be offered the control diet, a kibble, fed dry, only. The dogs assigned to Group 2 will receive the control diet, a kibble, fed dry and the test treat two hours after the removal of the control diet. The treat will be offered for a minimum of 1 hour. Treat consumption will be recorded for each animal once daily. The time the treats will be offered will be recorded in the raw data. The diet will be fed once daily for approximately a minimum of one hour. Dogs will be fed according to body condition and food amounts will be adjusted weekly if necessary to maintain body weight. Food consumption will be recorded daily.

### Test Phase (Day 1-27):

On Days 1-27, dogs assigned to Group 1 will be offered the control diet, a kibble, fed dry, only. The dogs assigned to Group 2 will continue to receive the control diet, a kibble, fed dry and the test treat two hours after the removal of the control diet. The treat will be offered for a minimum of 1 hour. Treat consumption will be recorded for each animal once daily. The time the treats will be offered will be recorded in the raw data. The diet will be offered for a minimum of one hour daily. Food consumption will be recorded daily.

On Day 27, the timing of offering the test treat may be adjusted to account for scoring time on the following day (dogs will be scored no more than +/− three (3) hours before the test treat would have been given if continued). The time the test treat is offered will be recorded in the raw data. The treats will be weighed before and after offering. Due to the length of time required for dental procedures, test treats will not be offered on dental procedures days.

### Dental Procedures:

### Anesthesia:

The dogs will be anesthetized for all scoring and cleaning procedures. Atropine administered subcutaneously will be administered on Days 0 and 28 prior to anesthetizing the dogs in order to aid in drying the mouth for scoring. Drugs and dosages administered will be recorded in the raw data.

### Cleaning Procedures:

At each cleaning procedure teeth will be professionally cleaned and polished by trained dental technicians. A soft toothbrush will be used to remove plaque and an ultrasonic scaler will be used to remove calculus. All subgingival, gingival, and coronal plaque and calculus will be removed. The teeth will then be polished with a glycerin/pumice polishing paste. After cleaning and polishing, teeth will be disclosed with 2% eosin. Repeat cleaning and polishing will be performed until it is determined a clean mouth model was established.

### Pre-Test Phase:

On Day 0, each animal will have saliva collected, halitosis measured, and gingivitis evaluated. Plaque will then be collected and after plaque collection the teeth will be cleaned and polished.

### Test Phase:

On Day 28, saliva will be collected, halitosis measured, and gingivitis and plaque will be scored. Plaque will be collected after being disclosed for scoring and prior to brushing for calculus evaluation.

The examination of teeth will include the buccal surfaces and both sides of the mouth. Review will be limited to these nine teeth: upper jaw—incisor 3 (I3), canine (C), premolar 3 (P3), premolar 4 (P4) and molar 1 (M1); lower jaw—canine (C), premolar 3 (P3), premolar 4 (P4), molar 1 (M1).

The dogs will be scored no more than +/− three (3) hours before the test treat would have been given if continued. The time the test treats are offered on the day prior to scoring (Day 27) may be adjusted if necessary to account for the +/− three (3) hour period. The test treat offering time will be recorded in the raw data.

### Dental Observations:

The presence or absence of inflammation, ulceration, or laceration anywhere in the oral cavity will be recorded for each dog during dental scoring on Days 0 and 28.

### Saliva Collection:

On Day 0 and 28 prior to administration of atropine, saliva samples will be collected using a DNAgenotek PERFORMAgene PG-100 oral collection device according to the manufacturer's guidelines.

The swab will be gently rubbed for 30 seconds in the areas where saliva naturally pools (in the right cheek pouch and under the tongue).

Swabs will then be placed into the manufacturer's tube and shaken vigorously 10 times to thoroughly mix samples.

Samples will be held in the collection tubes at room temperature until all collections are completed and then will be stored at −20° C. until shipment to a laboratory.

### Plaque Collection:

On Days 0 and 28, disclosed subgingival and supragingival plaque will be collected using a sterile dental probe from the following teeth on each side of the mouth: maxillary fourth premolar, first molar and mandibular fourth premolar and first molar. The plaque will be collected by taking the sterile dental probe (one probe per dog) and placing it on the gingival margin of the tooth and sweeping it along the base of the crown. The plaque sample will be placed into a 2.0 mL cryovial and stored at −80° C. until shipment to a laboratory.

### Clinical Observations/Measurements:

### Physical Examinations:

A veterinarian will give a complete physical examination to all dogs prior to the pre-test phase (Day −14). Each dog will be evaluated as to general health including oral health, body and hair coat condition, and comments will be recorded. Two milliliters of blood will be taken from each dog to test for BUN, PCV, and TP prior to treatment initiation if these data have not been updated within the last 6 months.

### Observations:

Qualified personnel will perform clinical observations twice daily. Examples of observations that will be recorded include abnormal stool quality, evidence of vomiting, abnormal behavior, poor coat quality, poor body condition, etc. Clinical laboratory diagnostic procedures will be performed as needed. Veterinary care will be given as appropriate to each individual animal.

### Dental Parameter Evaluation:

On Day 28, the dogs will be scored no more than +/− three (3) hours before the test treat would have been given if continued. The time the control and treat are offered on the day prior to scoring (Day 27) may be adjusted if necessary to account for the +/− three (3) hour period. The test treat offering time will be recorded in the raw data. The dogs will be presented to the blinded scorer randomly.

### Halitosis:

Oral malodor will be evaluated using an OralChroma prior to anesthetizing the dog. Readings will be obtained by holding the dog's mouth open for 3-5 seconds. The mouth is then closed for 30 seconds taking care to seal the lips fully. After 30 seconds, without unsealing the lips a 1 ml-syringe will be inserted between one of the upper canine teeth and the first premolar. A breath sample will be collected by pulling the plunger out to the end of the syringe. While still in the mouth, the syringe will be emptied and then filled again. The syringe will be removed from the mouth and slowly adjusted to the 1 ml mark. The end of the syringe is then wiped with a low lint delicate wipe. The gas sample will then be injected into the OralChroma for S-compound evaluation.

### Gingivitis:

Gingivitis, defined as the inflammation of the gums surrounding the teeth, will be evaluated by the modified gingival index based on Lobene et al. (1986). The MGI scoring system is as follows:

### Gingivitis Scoring Method

| Score | Degree of Inflammation |
|---|---|
| 0 | Absence of inflammation |
| 1 | Mild inflammation; slight change in color, |
| | little change in texture of any portion of the |
| | marginal or papillary gingival unit, no |
| | bleeding on probing |
| 2 | Mild inflammation; criteria as above but |
| | involving the entire marginal or papillary |
| | gingival unit, bleeding on probing within |
| | 30 seconds |
| 3 | Moderate inflammation; glazing, redness, |
| | edema, and/or hypertrophy of the marginal |
| | or papillary gingival unit, immediate |
| | bleeding on probing |
| 4 | Severe inflammation; marked redness, |
| | edema and/or hypertrophy of the marginal |
| | or papillary gingival unit, spontaneous |
| | bleeding, congestion, or ulceration |

Each tooth will be assigned a numerical score based on the degree of inflammation. The sum of the teeth scores will be divided by the number of teeth examined (18) to obtain a whole mouth mean gingivitis score for each animal.

### Plaque:

Plaque is defined as the soft, bacteria-rich layer that rapidly forms on the surface of the teeth. Plaque will be evaluated using a modification of the Quigley and Hein (1962) (Turesky, 1970) plaque index. The extent of plaque and plaque thickness will be determined by placing a disclosing agent on the teeth and rinsing the excess off with water. The whole tooth will be scored for the percentage of coronal surface covered with plaque and thickness of plaque. The score for each tooth is calculated by multiplying the coverage and thickness scores. The sum of the tooth scores will be divided by the number of teeth examined (18) to obtain a whole mouth mean plaque score for each animal.

### Plaque Scoring Method

| Score | % of Plaque Coverage |
|---|---|
| 0 | No plaque detected |
| 1 | 1-24 |
| 2 | 25-49 |
| 3 | 50-74 |
| 4 | 75-100 |

| Score | Plaque Thickness | Disclosing Agent Color |
|---|---|---|
| 1 | Light | Pink to Light Red |
| 2 | Medium | Red |
| 3 | Heavy | Dark Red |

### Calculus (Tartar):

Calculus is defined as the calcium salts secreted in saliva that are deposited on the surface of the teeth as a hard substance that is resistant to removal by chewing or brushing. Calculus is scored quantitatively on the whole buccal surface of the crown using modifications of a method developed by a method developed by Warrick-Gorrel.

When using the Warrick-Gorrel modified method the calculus is scored after the tooth is disclosed and a plaque score is obtained. Each tooth is then brushed using a toothbrush to remove the plaque, the teeth are rinsed with water and air-dried, then calculus is scored.

Each tooth is assigned a numerical score based on the percentage of calculus coverage and a thickness score (see calculus scoring method below). The score for each tooth is calculated by multiplying the coverage and thickness scores. The sum of the teeth scores will be divided by the number of teeth examined (18) to obtain a whole mouth mean calculus score for each animal.

### Calculus (Tartar) Scoring Method

| Score | % of Calculus Coverage |
|---|---|
| 0 | No calculus detected |
| 1 | 1-24 |
| 2 | 25-49 |
| 3 | 50-74 |
| 4 | 75-100 |
| Score | Calculus Thickness |
| 1 | Thin |
| 2 | Medium |
| 3 | Thick |

### Saliva and Plaque Analysis:

Salivary and plaque samples will be analyzed.

Raw data will be collected pertaining to: Plaque evaluation results

Calculus (tartar) evaluation results; Gingivitis evaluation results; Halitosis evaluation results; Saliva analysis (results will be reported directly to the Sponsor and not included in the report); Plaque analysis (results will be reported directly to the Sponsor and not included in the report); Food consumption; and Body weights.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

All issued patents, published patent applications, journal articles, and other publications, which are referred to herein, are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. An edible chew for preventing or inhibiting dental plaque accumulation and dental calculus formation in a non-human animal, the edible chew comprising:
a core comprising one or more of a polishing agent, a flavor additive, a vegetable oil, a humectant, a dental additive, a food additive, and water; and
an outer layer disposed on the core, the outer layer comprising astaxanthin.

2. The edible chew of claim 1, wherein the outer layer comprises from about 0.05 mg to about 10.0 mg of astaxanthin.

3. The edible chew of claim 2, wherein the outer layer comprises from about 0.2 mg to about 0.4 mg of astaxanthin.

4. The edible chew of claim 2, wherein the outer layer comprises from about 0.1 mg to about 0.2 mg of astaxanthin.

5. The edible chew of claim 2, wherein the outer layer comprises from about 1.5 mg to about 2.0 mg of astaxanthin.

6. The edible chew of claim 2, wherein the outer layer comprises from about 0.7 mg to about 1.1 mg of astaxanthin.

7. The edible chew of claim 2, wherein the outer layer comprises from about 2.0 mg to about 3.0 mg of astaxanthin.

8. The edible chew of claim 2, wherein the outer layer comprises from about 1.0 mg to about 1.5 mg of astaxanthin.

9. The edible chew of claim 2, wherein the outer layer comprises from about 3.5 mg to about 4.5 mg of astaxanthin.

10. The edible chew of claim 2, wherein the outer layer comprises from about 1.5 mg to about 2.5 mg of astaxanthin.

11. The edible chew of any one of claims 1-10, wherein the core comprises a polishing agent selected from sodium bicarbonate, calcium carbonate, dicalcium phosphate, calcium sulfate, silica, iron oxide, hydroxyapatite, aluminum oxide, perlite, plastic particles, or any combination thereof.

12. The edible chew of any one of claims 1-11, wherein the core comprises a polishing agent selected from calcium carbonate, dicalcium phosphate, sodium bicarbonate, or any combination thereof.

13. The edible chew of any one of claims 1-12, wherein the core comprises a flavor additive selected from spearmint, peppermint, wintergreen, sassafras, clove, sage, eucalyptus, marjoram, cinnamon, lemon, lime, grapefruit, coconut, orange, vanilla, rosemary, salt, chicken, honey, or any combination thereof.

14. The edible chew of any one of claims 1-13, wherein the core comprises a flavor additive selected from clove, cinnamon, vanilla, rosemary, salt, chicken, honey, or any combination thereof.

15. The edible chew of any one of claims 1-14, wherein the core comprises a vegetable oil selected from palm oil, soybean oil, canola oil, sunflower seed oil, flaxseed oil, peanut oil, cottonseed oil, palm kernel oil, coconut oil, olive oil, or any combination thereof.

16. The edible chew of any one of claims 1-15, wherein the core comprises a vegetable oil selected from canola oil, flaxseed oil, or any combination thereof.

17. The edible chew of any one of claims 1-16, wherein the core comprises a humectant selected from glycerin, sorbitol, or any combination thereof.

18. The edible chew of any one of claims 1-17, wherein the core comprises a dental additive selected from glucose oxidase, L-ascorbic acid phosphate, lactoperooxidase, tetrasodium pyrophosphate, or any combination thereof.

19. The edible chew of any one of claims 1-18, wherein the core comprises a food additive selected from cultured dextrose, gelatin, lecithin, rice flour, citric acid, or any combination thereof.

20. The edible chew of any one of claims 1-19, wherein each of the core and the outer layer independently comprise one or more binders and/or fillers.

21. The edible chew of any one of claims 1-20, wherein the outer layer includes an exterior surface facing away from the core, and wherein the exterior surface is substantially free of any protrusions configured to remove dental plaque and/or dental calculus from teeth of a subject.

22. A method of preventing or inhibiting dental plaque accumulation and dental calculus formation in a subject in need thereof, comprising administering to the subject an effective amount of astaxanthin, wherein the subject is a non-human animal.

23. The method of claim 22, wherein the astaxanthin is administered at a dose of from about 0.05 mg to about 10 mg at least once per day.

24. The method of claim 23, wherein the non-human animal has a weight of up to about 8.0 lbs, and wherein the astaxanthin is administered at a dose of from about 0.1 mg to about 0.5 mg at least once per day.

25. The method of claim 23, wherein the non-human animal has a weight of up to about 8.0 lbs, and wherein the astaxanthin is administered at a dose of from about 0.2 mg to about 0.4 mg once per day.

26. The method of claim 23, wherein the non-human animal has a weight of up to about 8.0 lbs, and wherein the astaxanthin is administered at a dose of from about 0.1 mg to about 0.2 mg twice per day.

27. The method of claim 23, wherein the non-human animal has a weight of from about 8.0 lbs to about 20.0 lbs, and wherein the astaxanthin is administered at a dose of from about 0.5 mg to about 2.5 mg at least once per day.

28. The method of claim 23, wherein the non-human animal has a weight of from about 8.0 lbs to about 20.0 lbs, and wherein the astaxanthin is administered at a dose of from about 1.5 mg to about 2.0 mg once per day.

29. The method of claim 23, wherein the non-human animal has a weight of from about 8.0 lbs to about 20.0 lbs, and wherein the astaxanthin is administered at a dose of from about 0.7 mg to about 1.1 mg twice per day.

30. The method of claim 23, wherein the non-human animal has a weight of from about 20.0 lbs to about 40.0 lbs, and wherein the astaxanthin is administered at a dose of from about 1.0 mg to about 3.5 mg at least once per day.

31. The method of claim 23, wherein the non-human animal has a weight of from about 20.0 lbs to about 40.0 lbs, and wherein the astaxanthin is administered at a dose of from about 2.0 mg to about 3.0 mg once per day.

32. The method of claim 23, wherein the non-human animal has a weight of from about 20.0 lbs to about 40.0 lbs, and wherein the astaxanthin is administered at a dose of from about 1.0 mg to about 1.5 mg twice per day.

33. The method of claim 23, wherein the non-human animal has a weight of greater than about 40.0 lbs, and wherein the astaxanthin is administered at a dose of from about 1.5 mg to about 5.0 mg at least once per day.

34. The method of claim 23, wherein the non-human animal has a weight of greater than about 40.0 lbs, and wherein the astaxanthin is administered at a dose of about 3.5 mg to about 4.5 mg once per day.

35. The method of claim 23, wherein the non-human animal has a weight of greater than about 40.0 lbs, and wherein the astaxanthin is administered at a dose of about 1.5 mg to about 2.5 mg twice per day.

36. The method of any one of claims 22-35, wherein the astaxanthin is administered as an edible chew.

37. The method of claim 36, wherein the edible chew comprises:
a core comprising one or more of a polishing agent, a flavor additive, a vegetable oil, a humectant, a dental additive, a food additive, and water; and
an outer layer disposed on the core, the outer layer comprising the effective amount of astaxanthin.

38. The method of any one of claims 22-37, wherein the non-human animal is at least 12 weeks old.

39. The method of any one of claims 22-38, wherein the non-human animal is a canine or a feline.

40. The method of any one of claims 22-39, wherein teeth of the non-human animal are cleaned prior to the administration of astaxanthin.

41. The method of any one of claims 22-40, wherein teeth of the non-human animal are substantially free of dental plaque prior to the administration of astaxanthin.

42. The method of any one of claims 22-41, wherein teeth of the non-human animal are substantially free of dental calculus prior to the administration of astaxanthin.

43. A method of preventing or inhibiting dental plaque accumulation in a subject in need thereof, comprising administering to the subject an effective amount of astaxanthin, wherein the subject is a non-human animal.

44. A method of preventing or inhibiting dental calculus formation in a subject in need thereof, comprising administering to the subject an effective amount of astaxanthin, wherein the subject is a non-human animal.

45. A method of preventing or inhibiting dental plaque accumulation and dental calculus formation in a subject in need thereof, comprising administering to the subject an effective amount of astaxanthin, wherein the astaxanthin is administered as an edible chew comprising:
a core comprising one or more of a polishing agent, a flavor additive, a vegetable oil, a humectant, a dental additive, a food additive, and water; and
an outer layer disposed on the core, the outer layer comprising the effective amount of astaxanthin,
wherein the subject is a non-human animal, and wherein the subject experiences an increase in salivary immunoglobulin A (sIgA) concentration following onset of administration.

46. A process for preparing an edible chew for preventing or inhibiting dental plaque accumulation and dental calculus formation in a non-human animal, the edible chew comprising a core comprising one or more of a polishing agent, a flavor additive, a vegetable oil, a humectant, a dental additive, a food additive, and water, and an outer layer disposed on the core, the outer layer comprising astaxanthin, the process comprising:
extruding the core; and
extruding the outer layer on the core to form the edible chew.

47. The process of claim 46, wherein the step of extruding the core and the step of extruding the outer layer are performed simultaneously.

48. The process of claim 46, wherein the step of extruding the outer layer is performed after the step of extruding the core.
